(19) United States
(12) Patent Application Publication
(10) Pub. No.: US 2009/0310740 A1
(43) Pub. Date: Dec. 17, 2009

(54) COMPUTED TOMOGRAPHY METHOD AND SYSTEM

(75) Inventors: PAUL E. LICATO, WAUWATOSA, WI (US); JIANG HSIEH, BROOKFIELD, WI (US); DARIN ROBERT OKERLUND, MUSKEGO, WI (US); UWE WIEDMANN, BUC (FR)

Correspondence Address: PETER VOGEL;GE HEALTHCARE, 20225 WATER TOWER BLVD., MAIL STOP W492, BROOKFIELD, WI, 53045, US

(73) Assignee: GENERAL ELECTRIC COMPANY, SCHENECTADY, NY (US)

(21) Appl. No.: 12/139,719

(22) Filed: Jun. 16, 2008

(51) Int. Cl. H05G 1/60; A61B 6/00

(52) U.S. Cl. 378/20; 378/4

(57) ABSTRACT

A method of computed tomography is disclosed herein. The method includes acquiring a first plurality of views during a first helical pass and acquiring a second plurality of views during a second helical pass. The method also includes generating an image based on both the first plurality of views and the second plurality of views.

### FIELD OF THE INVENTION

This disclosure relates generally to method and system of computed tomography that generates an image based on multiple sets of views.

### BACKGROUND OF THE INVENTION

Typically, in computed tomography (CT) systems, an x-ray source emits a fan-shaped x-ray beam or a cone-beam shaped x-ray beam toward a subject or object positioned on a support. The beam, after being attenuated by the subject, impinges upon a detector assembly. The intensity of the x-ray beam received at the detector assembly is typically dependent upon the attenuation of the x-ray beam by the subject. Each detector element of the detector assembly produces a separate electrical signal indicative of the attenuated x-ray beam received.

In known third generation CT systems, the x-ray source and the detector assembly are rotated on a gantry around the object to be imaged so that a gantry angle at which the fan-shaped x-ray beam intersects the object constantly changes. Data representing the strength of the received x-ray beam at each of the detector elements is collected across a range of gantry angles. The data are ultimately processed to form an image of the object.

Known CT systems may be used to collect multiple datasets in order to show how a subject changes over a period of time. For example, the data may be used to show a portion of the subject during different phases of a cardiac cycle or to show how an injected contrast agent perfuses into the subject's tissue over time. One problem is that known CT systems may expose the subject to an x-ray dose that is higher than necessary for procedures intended to show change over a period of time.

### BRIEF DESCRIPTION OF THE INVENTION

The above-mentioned shortcomings, disadvantages and problems are addressed herein which will be understood by reading and understanding the following specification.

In an embodiment, a method of computed tomography includes acquiring a first plurality of views during a first helical pass and acquiring a second plurality of views during a second helical pass. The method also includes generating an image based on both the first plurality of views and the second plurality of views.

In an embodiment, a method of computed tomography includes rotating an x-ray source mounted to a gantry around an object. The method includes translating the object in a first direction with respect to the gantry while rotating the x-ray source. The method includes acquiring a first incomplete set of views of a volume of interest of the object while translating the object in the first direction. The method includes translating the object in a second direction with respect to the gantry that is generally opposite to the first direction while rotating the x-ray source. The method includes acquiring a second incomplete set of views of the volume of interest of the object while translating the object in the second direction and generating an image based on both the first incomplete set of views and the second incomplete set of views.

In another embodiment, a computed tomography system includes a gantry adapted to rotate around an object, an x-ray source mounted to the gantry, a table for supporting the object, wherein the table is adapted to translate the object with respect to the gantry, and a controller connected to the gantry, the x-ray source, and the table. The controller is configured to acquire a first incomplete set of views of the object during a first helical pass and a second incomplete set of views of the volume of interest of the object during a second helical pass. The controller is also configured to generate an image based on both the first incomplete set of views and the second incomplete set of views.

Various other features, objects, and advantages of the invention will be made apparent to those skilled in the art from the accompanying drawings and detailed description thereof.

### BRIEF DESCRIPTION OF THE DRAWINGS

FIG. 1 is a schematic diagram illustrating a CT system in accordance with an embodiment;

FIG. 2 is a flow chart in accordance with an embodiment;

FIG. 3 is a flow chart in accordance with an embodiment; and

FIG. 4 is a flow chart in accordance with an embodiment.

### DETAILED DESCRIPTION OF THE INVENTION

In the following detailed description, reference is made to the accompanying drawings that form a part hereof, and in which is shown by way of illustration specific embodiments that may be practiced. These embodiments are described in sufficient detail to enable those skilled in the art to practice the embodiments, and it is to be understood that other embodiments may be utilized and that logical, mechanical, electrical and other changes may be made without departing from the scope of the embodiments. The following detailed description is, therefore, not to be taken as limiting the scope of the invention.

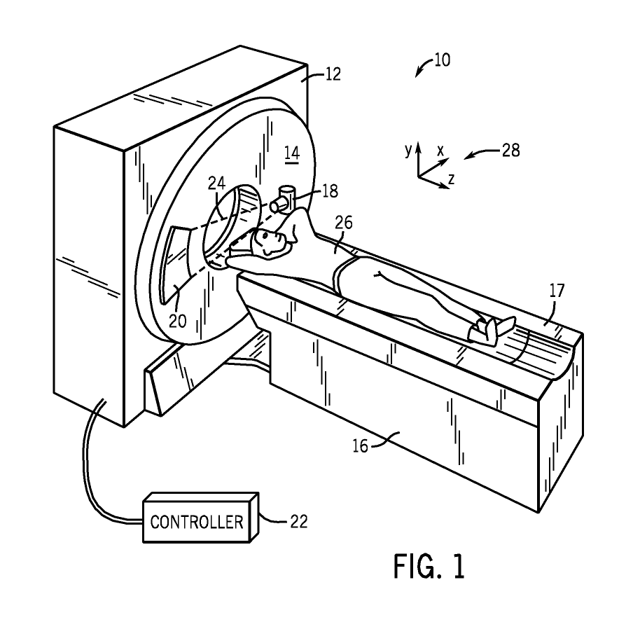

Referring to FIG. 1, a schematic representation of a computed tomography (CT) system 10 according to an embodiment is shown. The CT system 10 includes a gantry support 12, a gantry 14, a table 16, a moveable table portion 17, an x-ray source 18, a detector assembly 20, and a controller 22. The gantry 14 is configured to rotate within the gantry support 12. The gantry 14 is adapted to retain the x-ray source 18 and the detector assembly 20. The x-ray source 18 is configured to emit an x-ray beam 24 towards the detector assembly 20. The detector assembly 20 comprises a plurality of detector elements (not shown). Each of the plurality of detector elements produces an electrical signal that varies based on the intensity of the x-ray beam 24 received during a sampling interval. The table 16 is configured to support a subject or object 26 being scanned. The moveable table portion 17 is capable of translating the subject 26 in a z-direction with respect to the gantry 14 as indicated by coordinate axis 28. The controller 22 is configured to control the rotation of the gantry 14, the translation of the moveable table portion 17, and the activation of the x-ray source 18. The controller 22 is also configured to adjust a collimator aperture width. The term “collimator aperture width” includes the width of the x-ray beam 24 in generally the z-direction at generally a center of the gantry's 14 rotation. Additional details about the functioning of the controller 22 will be discussed hereinafter.

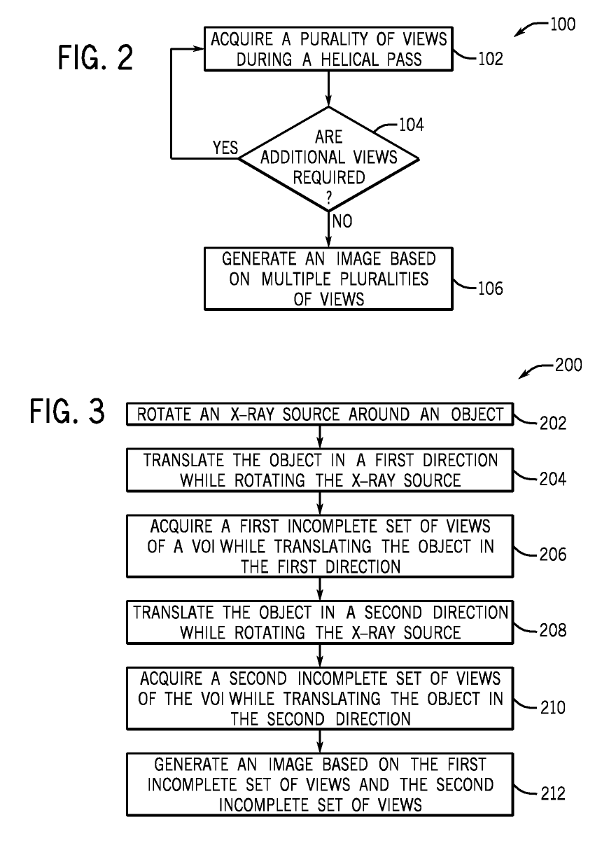

FIG. 2 is a flow chart illustrating a method 100 in accordance with an embodiment. The individual blocks 102-106 represent steps that may be performed in accordance with the method 100. The technical effect of the method 100 is the generation of a computed tomography image.

Referring to FIG. 1 and FIG. 2, at step 102, a first plurality of views of the object 26 are acquired during a first helical pass in a first direction with respect to the gantry 14. For the purposes of this disclosure, the term view is defined to include x-ray transmission data from the plurality of detector elements (not shown) that are acquired generally from a single angular position of the gantry 14 at a particular location in the z-direction with respect to the object 26. For the purposes of this disclosure, the term “helical pass” is defined to include collecting data with the computed tomography system 10 where the x-ray source 18 describes a helical or spiral path with respect to the object 26 being scanned. According to an embodiment, the object 26 may be advanced by moving the moveable table portion 17 in the z-direction with respect to the gantry 14 at the same time as the gantry 14 is rotating around the object 26 during the collection of x-ray transmission data. The combination of the translation of the moveable table portion 17 with the rotation of the gantry 14 results in the x-ray source 18 describing a spiral or helical path with respect to the object 26. It should be understood that according to additional embodiments, the translational movement between the object 26 and the gantry 14 could also be caused by translating the gantry 14 while keeping the object 26 stationary. Additionally, according to another embodiment, the translational motion between the object 26 and the gantry 14 could be caused by translating both the object 26 and the gantry 14.

According to an embodiment, a pitch of the helical acquisition may remain constant during the first helical pass of step 102. For the purposes of this disclosure, the term pitch is defined to include the ratio of the relative translational movement between the gantry 14 and the object 26 in one gantry rotation to the collimator aperture width. It should be understood that the term helical pass also includes computed tomography procedures where the pitch varies. For example, according to an embodiment, the pitch of the helical pass is varied by adjusting a translational velocity of the moveable table portion 17 with respect to the gantry 14. According to other embodiments, the pitch of the helical pass may also be varied by adjusting a rotational speed of the gantry 14 or by adjusting both the translation velocity of the moveable table portion 17 with respect to the gantry 14 and the rotational speed of the gantry 14.

In order to reconstruct an image of the object 26, a complete set of views is desired. The complete set of views is a limit that varies based on the design of the CT system. The complete set of views may be mathematically calculated under an ideal and hypothetical set of operating conditions. For the purposes of this disclosure, the complete set of views is defined to include the minimum number of views per gantry rotation needed to accurately reconstruct an image of the scanned volume assuming that the transmission data in each view is exact. According to one exemplary embodiment, a complete set of views may include 1000 views per gantry rotation for a CT system with a 1:1 pitch. The exact number of views in the complete set of views may vary based on the design of the CT system and the scan parameters. For the purposes of this disclosure, an incomplete set of views includes a set of views that does not contain all of the views included in the complete set of views. According to an embodiment, the controller 22 controls the x-ray source 18 in order to collect the odd numbered views during the first helical pass at step 102.

At step 104, the method 100 determines if additional views are required. If additional views are required, the method 100 cycles back to step 102 where a second plurality of views are acquired during a second helical pass. According to an embodiment, the object 26 is translated in a second direction with respect to the gantry 14 during the second helical pass. The second direction is generally opposite to the first direction of translation of the first helical pass. According to an embodiment, the controller 22 controls the x-ray source 18 in order to collect the even numbered views during the second helical pass.

If no additional views are required at step 104, the method 100 advances to step 106 where an image is generated based on multiple sets of views. For example, according to an embodiment, an image is generated by combining the first plurality of views acquired during the first helical pass at step 102 and the second plurality of views acquired during the second helical pass at step 102. Since the first plurality of views comprises the odd numbered views and the second plurality of views comprises the even numbered views, by combining the first plurality of views and the second plurality of views, a complete set of views may be formed. According to an embodiment, the first image is generated by using a filtered back-projection algorithm as is well-known by those skilled in the art. It should be understood that other embodiments may use a different reconstruction algorithm, such as an iterative reconstruction algorithm. Also, embodiments may acquire views in a different pattern. For example, embodiments may use more than two helical passes in order to collect the complete set of views.

According to an embodiment, the method 100 may cycle between step 102 and step 104 ten times. The direction of the helical pass at step 102 alternates with each helical pass as the method 100 cycles between steps 102 and 104. The subject 26 may be advanced in a positive z-direction with respect to the gantry 14 during the even helical passes and the subject 26 may be advanced in a negative z-direction with respect to the gantry 14 during the odd helical passes. By “shuttling” the subject 26 back-and-forth, the method 100 allows for the efficient acquisition of views of a volume of interest of the subject 26. According to another embodiment, the method 100 may collect a complete set of views during the odd helical passes and a complete set of views during the even passes. Then at step 106, the method 100 is able to generate a first image based on the views collected during the even helical passes and a second image based on the views collected during the odd helical passes. This acquisition technique may be beneficial for circumstances where the acquisition of views during each helical pass is gated on a physiological function of the subject 26, such as a cardiac cycle or a respiratory cycle.

According to another embodiment, views may be acquired at two or more different x-ray energy levels during each helical pass at step 102. For example, an embodiment may alternate between acquiring one or more views at a higher x-ray energy level and one or more views at a lower x-ray energy level during each helical pass. According to an exemplary embodiment, switching between the higher x-ray energy level and the lower x-ray energy level results in a first subset of views at the higher x-ray energy level and a second subset of views at a lower x-ray energy level during each helical pass. After multiple helical passes, a complete set of views for both the higher x-ray energy level and the lower x-ray energy level will have been acquired. It should be understood that additional embodiments may acquire views at more than two x-ray energy levels during each helical pass.

FIG. 3 is a flow chart illustrating a method 200 in accordance with an embodiment. The individual blocks 202-212 of the flow chart represent steps that may be performed in accordance with the method 200. The technical effect of method 200 is the generation of an image based on both a first incomplete set of views and a second incomplete set of views.

Referring to FIGS. 1 and 3, at step 202, the x-ray source 18 mounted to the gantry 14 is rotated at a rotational speed around the object 26. At step 204, the object 26 is translated at a translational velocity in a first direction with respect to the gantry 14 while the x-ray source is rotating around the object. According to an embodiment, the rotational speed of the x-ray source 18 rotating during step 202 may vary based on the translational velocity of the object 26 during step 204. For example, according to an exemplary embodiment, the rotational speed of the x-ray source 18 may increase as the translational velocity of the object 26 increases. Since the object 26 may be translated with respect to the gantry 14 from a stopped position during step 204, the object 26 would need to accelerate before reaching a peak translational velocity. By varying the rotational speed of the gantry 14 based on the translational velocity of the object 26, it is possible to acquire data in a helical acquisition of a desired pitch even though the object 26 is being translated at a variable translational velocity.

At step 206, a first incomplete set of views of a volume of interest (VOI) of the object 26 is acquired while translating the object 26 in the first direction. The first incomplete set of views is acquired by selectively activating the x-ray source 18 in order to acquire x-ray transmission data for only the desired views.

At step 208, the object 26 is translated in a second direction with respect to the gantry 14 while the x-ray source 18 is rotating around the object 26. According to an embodiment, the object 26 is translated in generally the opposite direction with respect to the gantry 14 as the object 26 was translated during step 204. At step 210, a second incomplete set of views of the volume of interest of the object 26 is acquired while translating the object in the second direction during step 208.

While acquiring the first incomplete set of views during step 206 and the second incomplete set of views during step 210, the intensity of the x-ray source 18 may be varied. The intensity of the x-ray source 18 may be varied to provide more uniform noise characteristics or to reduce the x-ray dose to which the object 26 is exposed. The intensity of the x-ray source 18 may be adjusted by altering an amount of current that is supplied to the x-ray source 18. The intensity of the x-ray source 18 may vary based on the translational velocity of the object 26 with respect to the gantry 14. According to an embodiment, the intensity of the x-ray source 18 may vary as a function of the translational velocity of the object 26 with respect to the gantry 14. According to another embodiment, the intensity of the x-ray source 18 may vary based on a gantry angle of the gantry 14 with respect to the gantry support 12. In cases where the subject 26 is being imaged, a lower intensity x-ray beam 24 may be used in the anterior-posterior direction compared to the lateral direction. Therefore, according to an embodiment, the x-ray source 18 may be configured to emit the x-ray beam 24 with a lower intensity for gantry angles where the x-ray beam 24 enters the subject 26 in generally the anterior-posterior direction compared to gantry angles where the x-ray beam 24 enters the subject 26 in generally the lateral direction. It should be appreciated that additional embodiments may utilize different methods of varying the intensity of the x-ray beam 24.

At step 212, an image is generated based on the first incomplete set of views and the second incomplete set of views. According to an embodiment, a filtered backprojection reconstruction algorithm is used to generate the image. However, additional embodiments may use other types of reconstruction algorithms. It should be appreciated that additional embodiments may collect more than two incomplete sets of views before generating an image.

According to another embodiment, the collimator aperture width may be adjusted during the method 200. For example, while the object 26 is being translated in a first direction during step 204, the x-ray beam 24 may expose a portion of the object 26 other than the volume of interest at both the start of the translation and at the end of the translation. Therefore, an embodiment may activate a shutter of the collimator (not shown) to minimize the amount of the x-ray beam 24 that extends beyond the volume of interest in the direction of the translation. In a similar manner, the shutter of the collimator may be activated during the second translation of step 208, or during any subsequent translation steps according to other embodiments.

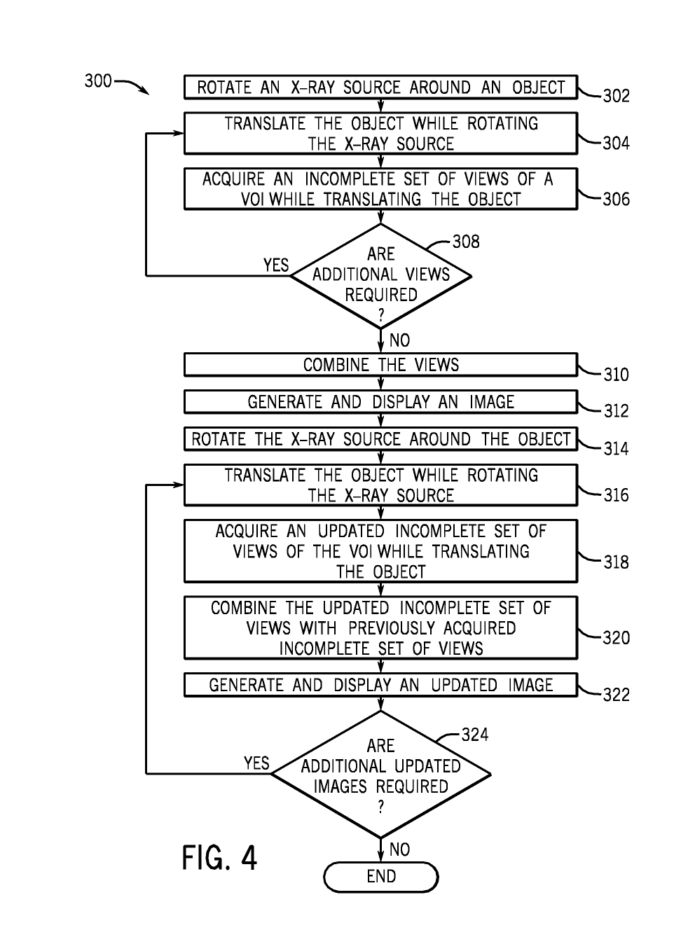

FIG. 4 is a flow chart illustrating a method 300 in accordance with an embodiment. The individual blocks 302-324 represent steps that may be performed in accordance with the method 300. The technical effect of the method 300 is the generation of a computed tomography image

Referring to FIGS. 1 and 4, at step 302, the x-ray source 18 mounted to the gantry 14 is rotated around the object 26. At step 304, the object 26 is translated in a first direction with respect to the gantry 14 while the x-ray source 18 is rotating around the object 26. The combination of the rotation of the x-ray source at step 302 and the translation of object at step 304 causes the x-ray source 18 to describe a helical path with respect to the object 26 during a first helical pass. At step 306, the x-ray source 18 is activated during a portion of the time while the x-ray source 18 is rotating and the object 26 is being translated, and a first incomplete set of views of a volume of interest (VOI) of the object 26 is acquired. According to an exemplary embodiment where a complete set of views equals 1,000 views per rotation, only 2 out of every 20 views may be acquired during each helical pass. For example, views 1-2, 21-22, 41-42, . . . , 981-982 are acquired during the first helical pass. If only 2 out of every 20 views are acquired, the x-ray source 18 may only need to be activated for 10% of the time. It should be appreciated that it would be possible to acquire an incomplete set of views in a manner other than the one described. For example, alternate embodiments may acquire a different number of views or acquire views in a different order from the exemplary embodiment.

At step 308, the controller 22 determines if additional views are required. If additional views are required, the method 300 returns to step 304. At step 304, the object 26 is translated in the opposite direction from the previous helical pass. By switching the direction of translation of each successive helical pass it is possible to cover the same volume of interest (VOI) with each helical pass. At step 306, a second incomplete set of views of the volume of interest (VOI) of the object 26 is acquired. According to an exemplary embodiment, the second incomplete set of views includes views 3-4, 23-24, 43-44, . . . , 983-984. According to this embodiment, the computed tomography system 10 would take a total of 10 helical passes in order to collect enough information to generate an image. According to an embodiment, the third helical pass would collect views 5-6, 25-26, 45-46, . . . , 985-986. If the pattern of advancing the views collected on each helical pass continues in the same manner as described above, the method 300 would cycle through the loop between steps 304 and 308 ten times before a complete set of views was collected. It should be understood that according to an embodiment, the object 26 is translated in the opposite direction during each successive helical pass. For example, during helical passes 1, 3, 5, 7, and 9 the object 26 may be translated in generally the positive z-direction and during helical passes 2, 4, 6, 8, and 10 the object 26 may be translated in generally the negative z-direction.

After the method 300 has cycled through the loop between steps 304 and 308 ten times, the incomplete sets of views acquired during step 306 are combined during step 310. At step 312, an image is generated and displayed based on the combined sets of incomplete views.

At step 314, the x-ray source 18 is rotated around the object 26. According to an embodiment, the x-ray source may keep rotating from step 302 until step 314. At step 316, the object 26 is translated while x-ray source 18 is rotating. At step 318, the x-ray source 18 is activated during a portion of the time while the x-ray source 18 is rotating and the object 26 is being translated, and an updated incomplete set of views is acquired. Since a complete set of views had already been acquired, the updated incomplete set of views acquired at step 318 duplicates some of the views that were previously acquired. However, the views acquired at step 318 represent the object 26 at a later point in time. For example, according to one embodiment, the eleventh helical pass collects the same plurality of views as were collected during the first helical pass. In other words, the eleventh helical pass collects transmission data from views 1-2, 21-22, 41-42, . . . , 981-982. At step 320, the updated incomplete set of views are combined with the previously acquired incomplete sets of views in order to form an updated complete set of views. For example, according to an embodiment, the eleventh incomplete set of views are combined with the second through tenth incomplete sets of views acquired during step 306, and an updated image is generated and displayed at step 322. If additional updated images are required at step 324, the method 300 loops back to step 316 and another updated incomplete set of views is acquired. The updated incomplete set of views may then be combined with previously acquired incomplete sets of views from either step 306 or step 318 in order to form a complete set of views so that an updated image can be generated. According to an embodiment, the method 300 acquires the updated incomplete sets of views at step 320 in the same order as the incomplete sets of views were acquired-during steps 304-308. If no additional updated images are required, the method 300 ends. It should be appreciated by one skilled in the art that the updated incomplete sets of views may be acquired and/or combined in an order different from the one described above according to additional embodiments.

The method 300 may be used to show how an the object 26 changes with time. For example, the method 300 may be used to acquire and display perfusion or computed tomography angiography information. For example, the volume of interest may include a portion of the subject's 26 vasculature during an angiography study.

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims.

We claim:

1. A method of computed tomography comprising:
acquiring a first plurality of views during a first helical pass;
acquiring a second plurality of views during a second helical pass; and
generating an image based on both the first plurality of views and the second plurality of views.

2. The method of claim 1, further comprising acquiring a physiological signal and using the physiological signal to initiate said acquiring the first plurality of views.

3. The method of claim 2, wherein said acquiring the physiological signal comprises acquiring an electrocardiograph.

4. The method of claim 1, wherein said acquiring the first plurality of views comprises switching an x-ray source from a first x-ray energy level to a second x-ray energy level during the first helical pass.

5. The method of claim 4, wherein said acquiring the first plurality of views further comprises acquiring a first subset of views at generally the first x-ray energy level and acquiring a second subset of views at generally the second x-ray energy level.

6. The method of claim 5, wherein said acquiring the second plurality of views comprises acquiring a third subset of views at generally the first x-ray energy level and acquiring a fourth subset of views at generally the second x-ray energy level.

7. A method of computed tomography comprising:
rotating an x-ray source mounted to a gantry around an object;
translating the object in a first direction with respect to the gantry while said rotating the x-ray source;
acquiring a first incomplete set of views of a volume of interest of the object while said translating the object in the first direction;
translating the object in a second direction with respect to the gantry while said rotating the x-ray source, the second direction being generally opposite to the first direction;
acquiring a second incomplete set of views of the volume of interest of the object while said translating the object in the second direction; and
generating an image based on both the first incomplete set of views and the second incomplete set of views.

8. The method of claim 7, wherein said rotating the x-ray source comprises rotating the x-ray source at a rotational speed that varies.

9. The method of claim 8, wherein said rotating the x-ray source at a rotational speed that varies comprises rotating the x-ray source at a rotational speed that varies based on a translational velocity of the object with respect to the gantry.

10. The method of claim 7, further comprising varying an intensity of an x-ray beam emitted from the x-ray source during said acquiring the first incomplete set of views.

11. The method of claim 10, wherein said varying the intensity of the x-ray beam comprises varying the intensity of the x-ray beam based on a translational velocity of the object with respect to the gantry.

12. The method of claim 10, wherein said varying the intensity of the x-ray beam comprises varying the intensity of the x-ray beam based on a gantry angle.

13. The method of claim 7, further comprising adjusting a collimator based on the relative position of the object with respect to the gantry to reduce the amount of an x-ray beam contacting the object outside of the volume of interest.

14. The method of claim 7, further comprising translating the object in the first direction with respect to the gantry a second time after said translating the object in the second direction.

15. The method of claim 14, further comprising acquiring a third incomplete set of views while said translating the object in the first direction with respect to the gantry the second time.

16. The method of claim 15, further comprising generating a second image based on both the second incomplete set of views and the third incomplete set of views in order to show a change that occurred in the object over a period of time.

17. The method of claim 16, further comprising analyzing the first image and the second image as part of either an angiography study or a perfusion study.

18. A computed tomography system comprising:
a gantry adapted to rotate around an object;
an x-ray source mounted to the gantry;
a table for supporting the object, wherein the table is adapted to translate the object with respect to the gantry; and
a controller connected to the gantry, the x-ray source, and the table;
wherein the controller is configured to acquire a first incomplete set of views of the object during a first helical pass and a second incomplete set of the object during a second helical pass; and
wherein the controller is configured to generate an image based on both the first incomplete set of views and the second incomplete set of views.

19. The computed tomography system of claim 18, wherein the controller is further configured to initiate the acquisition of the first incomplete set of views based on a physiological signal.

20. The computed tomography system of claim 18, wherein the controller is further configured to acquire a third incomplete set of views during a third helical pass.

21. The computed tomography system of claim 20, wherein the controller is further configured to generate a second image based on both the second incomplete set of views and the third incomplete set of views.

22. The computed tomography system of claim 21, wherein the controller is further configured to display the second image as part of an angiography study or as part of a perfusion study.

23. The computed tomography system of claim 21, wherein the controller is further configured to identify a change between the first image and the second image as part of either a perfusion study or an angiography study.
